(19) United States
(12) Patent Application Publication
(10) Pub. No.: US 2015/0196746 A1
(43) Pub. Date: Jul. 16, 2015

(54) NEEDLE ARRAY TRANSDERMAL ABSORPTION SHEET AND METHOD FOR MANUFACTURING NEEDLE ARRAY TRANSDERMAL ABSORPTION SHEET

(71) Applicant: FUJIFILM Corporation, Tokyo (JP)

(72) Inventors: Shotaro OGAWA, Ashigarakami-gun (JP); Aya MOCHIZUKI, Ashigarakami-gun (JP); Ryoichi NEMORI, Ashigarakami-gun (JP); Yanlong CHE, Ashigarakami-gun (JP)

(73) Assignee: FUJIFILM Corporation, Tokyo (JP)

(21) Appl. No.: 14/601,302

(22) Filed: Jan. 21, 2015

Related U.S. Application Data
(62) Division of application No. 13/247,476, filed on Sep. 28, 2011.

(30) Foreign Application Priority Data
Sep. 29, 2010 (JP) 2010-219176
Mar. 7, 2011 (JP) 2011-049251

(51) Int. Cl. A61M 37/00

(52) U.S. Cl. CPC A61M 37/0015; A61M 2037/0053; A61M 2207/00; A61M 2037/0023

(57) ABSTRACT

A needle array transdermal absorption sheet to be attached onto a skin for supplying a drug into the skin, includes: a plurality of needle portions each having a tapered shape, each of the needle portions including a needle having a conical or pyramidal shape and a body part which has a columnar shape and whose end surface is connected to a base of the needle; a sheet portion having a flat-plate shape; and a plurality of frustum portions each having a frustum shape, the frustum portions which are arranged on a surface of the sheet portion in a manner that perimeters of larger bases of adjacent frustum portions are in contact with each other on the surface of the sheet portion, and smaller bases of which are respectively connected to the body parts of the needle portions.

This application is a divisional of U.S. application Ser. No. 13/247,476, filed Sep. 28, 2011, which claims priority to JP 2010-219176, filed Sep. 29, 2010, and JP 2011-049251, filed Mar. 7, 2011, each of which is incorporated herein by reference in its entirety.

### BACKGROUND OF THE INVENTION

1. Field of the Invention

The present invention relates to a needle array transdermal absorption sheet that has drug-containing needle-shaped protrusions formed on the sheet and supplies the drug into a skin when the sheet is attached onto the skin, and a method for manufacturing the needle array transdermal absorption sheet.

2. Description of the Related Art

In recent years, attention has been directed to a needle array transdermal absorption sheet having drug-containing biodegradable microscopic needles formed on a surface of the sheet. When the sheet is attached onto the skin, the microscopic needles are inserted into the skin and absorbed in the skin, and the drug contained in the microscopic needles is supplied into the skin.

As a method for manufacturing such a needle array transdermal absorption sheet, there has been a known method in which a resin solution is poured into a mold having a large number of recesses to transfer the shape of the mold to the resin solution. In this method, a needle array transdermal absorption sheet having drug-containing microscopic needles can be produced by forming the microscopic needles made of a biocompatible, biodegradable resin solution to which the drug is added in advance.

Japanese Patent Application Laid-Open Nos. 2010-57704 and 2009-61219, for example, propose needle array transdermal absorption sheets. Japanese Patent Application Laid-Open No. 2010-57704 describes a needle-shaped member having a stepped (stepwise) inclined surface. The stepped portion separates the inclined surface on the sharpened portion side from the inclined surface on the root side, and the inclination angle of the inclined surface on the sharpened portion side differs from the inclination angle of the inclined surface on the root side. Japanese Patent Application Laid-Open No. 2010-57704 states that defects caused when the needle-shaped member having the microscopic structure is shaped and the shape is transferred can be suppressed.

Japanese Patent Application Laid-Open No. 2009-61219 describes a microscopic needle array so configured that each needle is connected to a hemispherical base. Japanese Patent Application Laid-Open No. 2009-61219 states that the configuration allows microscopic needles having a pin-holder shape (needle point holder shape) to be manufactured in quantity at low cost.

### SUMMARY OF THE INVENTION

However, for example, as shown in FIG. 11, which is a schematic view showing a state in which a needle array transdermal absorption sheet of related art is attached onto a skin, when the needle array transdermal absorption sheet of related art, which has a structure in which conical needle portions 1 are connected to a sheet portion 3, is attached to a skin 100, convex portions of convexo concaves of the surface of the skin 100 push the sheet portion 3 back. In this case, the needle portions 1 cannot be reliably inserted into the skin 100 to a point where the root or the sheet-side end of each of the needle portions 1 comes into contact with the skin 100. As a result, the drug in the root of each needle portion 1 is not absorbed in the skin 100, disadvantageously resulting in waste of the expensive drug.

There is another disadvantage: When each needle portion 1 has a conical or pyramidal shape, if the needle portion 1 comes off the skin 100 even by a small amount, a space is created between the needle portion 1 and the skin 100. Since no friction is produced between the needle portion 1 and the skin 100 with the space therebetween, the needle portion 1 easily comes off the skin 100.

Further, in the case of the needle-shaped member described in Japanese Patent Application Laid-Open No. 2010-57704, the side surface of each root portion, on which the corresponding sharp portion is formed, is not in contact with the side surfaces of the adjacent root portions. When the needle-shaped members are manufactured in a molding process, a drug-containing liquid is left in the mold, specifically, on a flat portion corresponding to the gap between adjacent root portions, that is, the drug-containing liquid is left on portions corresponding to the sheet. When the needle-shaped members are eventually formed, the expensive drug is also contained in the sheet, resulting in waste of the expensive drug.

Moreover, each root portion, on which a sharp portion is formed, has a surface on which the sharp portion is formed. Since the surface is parallel to the skin, the patient probably feels pain when the needle portion is inserted into the skin. Further, since the sharp portion and the root portion of each needle portion have pyramidal shapes, the needle portion easily comes off the skin, as described above.

Moreover, in the case of the needle-shaped member described in Japanese Patent Application Laid-Open No 2010-57704, the root portion thereof may also be inserted into the skin. In this case, convex portions of convexo concaves of the surface of the skin push the sheet back, and the needle-shaped members cannot be reliably inserted into the skin to a point where the root or the support substrate-side end of each of the needle portions comes into contact with the skin. In this case, the drug in the root or the support substrate-side end of each needle portion is not absorbed in the skin, disadvantageously resulting in waste of the expensive drug.

In the microscopic needle array described in Japanese Patent Application Laid-Open No. 2009-61219, in which each needle is connected to a hemispherical base, the side surface of a hemispherical base is not in contact with the side surfaces of the adjacent hemispherical bases. When the microscopic needles are manufactured in a molding process, a drug-containing liquid is left in the mold, specifically, on a flat portion corresponding to the gap between hemispherical bases, that is, the drug-containing liquid is left on portions corresponding to the sheet, as described above. When the microscopic needles are eventually formed, the expensive drug is also contained in the sheet, resulting in waste of the expensive drug.

Further, since each microscopic needle has a cylindrical shape, it is difficult to insert the needle into the skin. Moreover, when the interval at which the microscopic needles are arranged is too small, it is more difficult to insert the needles into the skin, whereas when the interval is too large, the amount of supplied drug decreases and hence the action thereof is insufficient. Japanese Patent Application Laid-Open No. 2009-61219 has no description or suggestion about the problem or discloses no interval that can solve the problem.

The present invention has been made in view of the circumstances described above. An object of the present invention is to provide a needle array transdermal absorption sheet and a method for manufacturing the needle array transdermal absorption sheet, the needle array transdermal absorption sheet whose needle portions, which are needles formed to be inserted into a skin, can be reliably inserted into the skin to their roots each of which is an end on a sheet portion side of respective needle portions so that a drug is not wasted and the needle resists coming off the skin.

The objects of the present invention can be achieved by the following aspects of the present invention:

That is, a needle array transdermal absorption sheet according to an aspect of the present invention is a sheet to be attached onto a skin for supplying a drug into the skin, including: a plurality of needle portions each having a tapered shape, each of the needle portions including a needle having a conical or pyramidal shape and a body part which has a columnar shape and whose end surface is connected to a base of the needle; a sheet portion having a flat-plate shape; and a plurality of frustum portions each having a frustum shape, the frustum portions which are arranged on a surface of the sheet portion in a manner that perimeters of larger bases of adjacent frustum portions are in contact with each other on the surface of the sheet portion, and smaller bases of which are respectively connected to the body parts of the needle portions.

According to the configuration described above, when the needle portions are inserted into the skin, convex portions of convexo concaves of the skin can enter the space between adjacent frustum portions, and thus, the needle portions can be reliably inserted into the skin to a point where the root of each needle portion, which is a sheet-side end of each needle portion, comes into contact with the skin. The expensive drug will therefore not be wasted. Further, since the peripheries of the larger bases adjacent frustum portions are in contact with each other, when the needle array transdermal absorption sheet of the present invention is manufactured in a molding process, the portions corresponding to the portions where the peripheries of the larger bases of the frustum portions are in contact with each other do not become flat in the mold, whereby a liquid containing the expensive drug is not left on the portion. Thus, waste of expensive drug can be prevented.

A needle array transdermal absorption sheet according to another aspect of the present invention is a sheet A needle array transdermal absorption sheet to be attached onto a skin for supplying a drug into the skin, including: a plurality of needle portions each having a tapered shape, each of the needle portions including a needle having a pencil-like shape and a body part which has a columnar shape and whose end surface is connected to a base of the needle; a sheet portion having a flat-plate shape; and a plurality of frustum portions each having a frustum shape, the frustum portions which are arranged on a surface of the sheet portion in a manner that perimeters of larger bases of adjacent frustum portions are in contact with each other on the surface of the sheet portion, and smaller bases of which are respectively connected to the body parts of the needle portions.

According to the configuration described above, each needle portion having a pencil-like shape is readily inserted into the skin but resists coming off the skin. Further, each needle portion having a pencil-like shape is stably supported by the corresponding frustum portion having a truncated conical or pyramidal shape without sideways movement when the needle portion is inserted into the skin and the stress produced at the time of insertion is widely distributed. Further, when the needle portions are inserted into the skin, convex portions of convexo concaves of the skin can enter the space between adjacent frustum portions, whereby the needle portions can be reliably inserted into the skin to a point where the root of each needle portion, which is a sheet-side end of each needle portion, comes into contact with the skin. The expensive drug will therefore not be wasted. Further, since the peripheries of the larger bases of adjacent frustum portions are in contact with each other, when the needle array transdermal absorption sheet of the present invention is manufactured in a molding process, the portions corresponding to the portions where the peripheries of the larger bases of the frustum portions are in contact with each other will not be flat in the mold, whereby a liquid containing the expensive drug is not left on the portion. Thus, waste of the expensive drug can be prevented.

In the needle array transdermal absorption sheet according to the aspects of the present invention, an angle β between a side surface of each of the frustum portions and a plane parallel to the surface of the sheet portion falls within a range of 20° to 60°.

If the angle β is too large, a patient may feel pain when the needle portions are inserted into the skin so deeply that the frustum portions are also inserted into the skin. According to the configuration described above, because the angle β is not too large, such a problem can be prevented. Further, setting the angle β within the range described above allows a sufficiently large space to be formed between the side surfaces of adjacent frustum portions when the needle portions are inserted into the skin, whereby convex portions of convexo concaves of the skin can enter the space and the needle portions can be reliably inserted into the skin to a point where the roots of the needle portions come into contact with the skin.

Further, in the needle array transdermal absorption sheet according to the aspects of the present invention, a height of each of the frustum portions is a value within a range of 0.1 mm to 0.5 mm. According to the configuration described above, when the needle portions are inserted into the skin, a larger space can be formed between the side surfaces of adjacent frustum portions, and thus, convex portions of convexo concaves of the skin can enter the space and the needle portions can be more reliably inserted into the skin to a point where the roots of the needle portions come into contact with the skin.

Further, in the needle array transdermal absorption sheet according to the aspects of the present invention, each of the needle portions having a tapered shape (forward converging shape) includes the needle having a circular conical shape and the body part having a cylindrical columnar shape, and each of the frustum portions has a truncated pyramidal shape.

Further, in the needle array transdermal absorption sheet according to the aspects of the present invention, each of the needle portions having a pencil-like shape includes the needle having a circular conical shape and the body part having a cylindrical columnar shape, and each of the frustum portions has a truncated pyramidal shape.

Further, in the needle array transdermal absorption sheet according to the aspects of the present invention, wherein when the larger bases of the frustum portions are placed in a horizontal plane, a normal vector to each of the frustum portion is not parallel to a vertical direction.

A method for manufacturing a needle array transdermal absorption sheet according to a further aspect of the present invention is a method for manufacturing the needle array transdermal absorption sheets according to any one of the aspects, the method including: injecting a first polymer dissolved liquid containing the drug into a space of a mold, the space having a shape identical to a shape of the needle array transdermal absorption sheet; filling the space in the mold with the first polymer dissolved liquid to an end of the space by pressurizing the mold into which the first polymer dissolved liquid has been injected to remove air bubbles from a portion where the first polymer dissolved liquid has been injected; drying and shrinking the first polymer dissolved liquid by heating the first polymer dissolved liquid so that all the first polymer dissolved liquid in the space in the mold is positioned in portions closer to spaces corresponding to the needles than portions corresponding to portions where the peripheries of the larger bases of the frustum portions are in contact with each other; injecting a second polymer dissolved liquid into the space in the mold; solidifying the first polymer dissolved liquid and the second polymer dissolved liquid by heating the first polymer dissolved liquid and the second polymer dissolved liquid; and separating and removing the solidified first and second polymer dissolved liquids from the mold.

In the mold used in the method described above, since the peripheries of the larger bases of adjacent frustum portions are in contact with each other, there are no flat portions on which the expensive liquid containing the drug are left in the contact portions. In addition, the method includes the process of “drying and shrinking the first polymer dissolved liquid so that all the first polymer dissolved liquid in the space in the mold is positioned in portions closer to spaces corresponding to the needles than portions corresponding to portions where the peripheries of the larger bases of the frustum portions are in contact with each other.” Thus, the liquid containing the expensive drug therefore is not left on the sheet portion.

According to the needle array transdermal absorption sheet of the present invention, the needle portions, each of which is a needle formed to be inserted into a skin, can be reliably inserted into the skin.

### BRIEF DESCRIPTION OF THE DRAWINGS

FIG. 1 is a schematic view showing a state in which a needle array transdermal absorption sheet according to an embodiment of the invention is attached onto a skin;

FIGS. 2A and 2B show an example of the arrangement of cone-type needle portions and frustum portions (truncated tapered portions);

FIGS. 3A and 3B are plan views of examples of the arrangement of the cone-type needle portions and frustum portions;

FIGS. 4A and 4B show an example of the arrangement of square pyramid-type needle portions and frustum portions;

FIGS. 5A and 5B show an example of the arrangement of combination-type needle portions and frustum portions;

FIGS. 6A and 6B are plan views, front views, and side views of combination-type needle portions and frustum portions;

FIGS. 7A to 7F show processes for manufacturing the needle array transdermal absorption sheet;

FIG. 8A to 8D show part of the processes for manufacturing the needle array transdermal absorption sheet;

FIGS. 9A to 9C show part of the processes for manufacturing the needle array transdermal absorption sheet;

FIG. 10 shows reference characters that designate dimensional parameters of a variety of portions of the needle array transdermal absorption sheet; and

FIG. 11 is a schematic view showing a state in which a needle array transdermal absorption sheet of related art is attached onto a skin.

### DETAILED DESCRIPTION OF THE PREFERRED EMBODIMENTS

An embodiment of the present invention will be described below in detail with reference to the accompanying drawings. The portions having the same reference characters throughout the drawings are similar elements having similar functions. In the present specification, a numerical range expressed by using “-” is intended to include the upper and lower limits of the numerical range.

### <Configuration of Needle Array Transdermal Absorption Sheet>

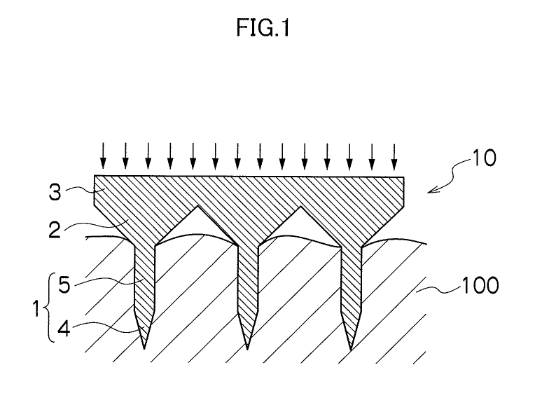

A needle array transdermal absorption sheet according to an embodiment of the present invention will be described with reference to the drawings. FIG. 1 is a schematic view showing a state in which the needle array transdermal absorption sheet according to the embodiment of the invention is attached onto a skin.

A needle array transdermal absorption sheet 10 according to the present invention primarily includes needle portions 1, each of which has a pencil-like shape, frustum portions (truncated tapered portions) 2, each of which has a truncated conical or pyramidal shape, and a sheet portion 3 having a flat-plate shape, as shown in FIG. 1.

Each of the needle portions 1 primarily has a needle 4 having a conical or pyramidal shape and a body part 5 having a cylindrical or rectangular columnar shape, and the bottom surface of the needle 4 is connected to an end surface of the body part 5.

The end surface of the body part 5 that is not connected to the needle 4 is connected to a smaller base (upper base) of the frustum portion 2, and a larger base (lower base) of the frustum portion 2 is connected to one surface of the sheet portion 3.

A plurality of thus configured frustum portions 2 connected to the respective needle portions 1 are formed on one surface of the sheet portion 3, and the side surfaces of adjacent frustum portions 2 are in contact with each other on the sheet portion 3.

The needle portions 1 and the frustum portions 2 are made of a biocompatible, biodegradable material, such as a polysaccharide, and in particular, the needle portions 1 contain a drug.

The needle portions 1 are preferably made of a material that not only tends to decompose in a living body (biodegradable material) but also has biocompatibility (biocompatible material). Specifically, any of the following materials can be used: gelatin, agarose, pectin, gellan gum, carageenan, xanthan gum, alginic acid, dextrin, dextran, starch, pullulan, cellulose, hyaluronic acid, chondroitin sulfuric acid, and other saccharides and gelled polymers.

The “drug” used herein collectively refers to a material that has efficacy providing an advantageous effect on the human body, such as insulin, nitroglycerin, vaccines, antibiotics, antasthmatic drugs, analgesics and narcotics for medicinal purposes, local anesthetics, antianaphylactic agents, dermatologic agents, sleep inducing drugs, vitamins, smoking-cessation assisting drugs, protein drags and cosmetic drugs.

The sheet portion 3 may be made of a biodegradable material (such as a polysaccharide) or may be made of a plastic resin.

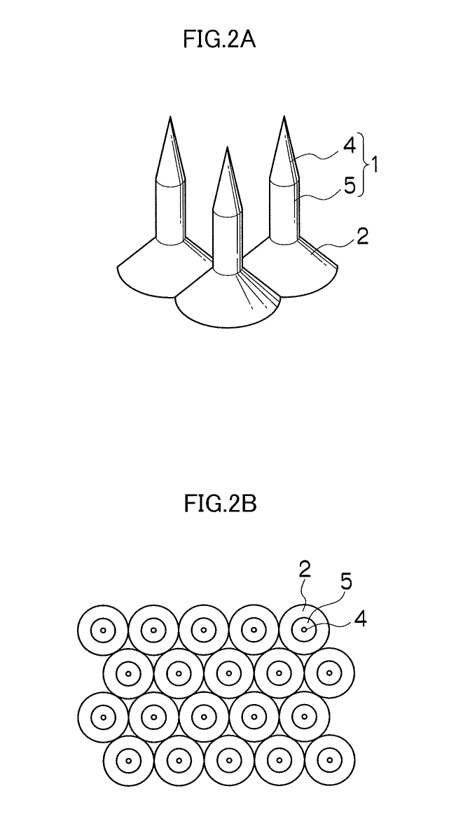

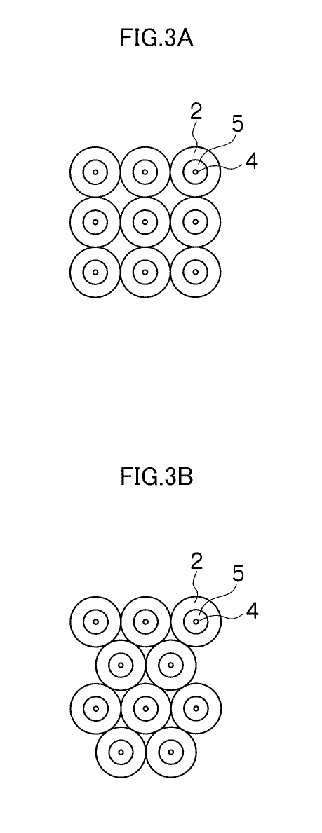

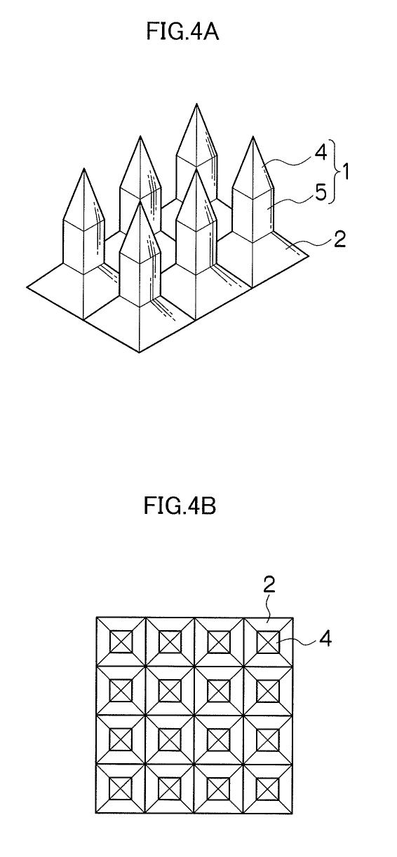

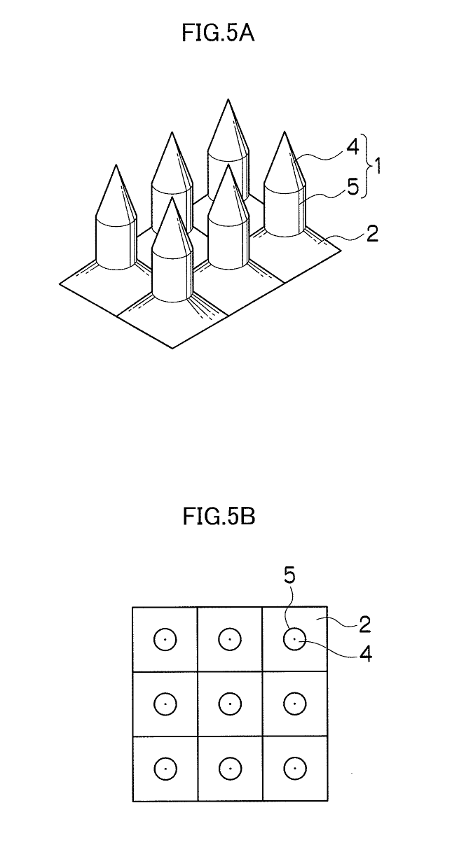

The arrangement of the needle portions 1 and the frustum portions 2 over the sheet portion 3 will next be described with reference to FIGS. 2A to 6B. FIGS. 2A and 2B show an example of the arrangement of cone-type needle portions 1 and frustum portions 2. FIGS. 3A and 3B are plan views of examples of the arrangement of the cone-type needle portions 1 and frustum portions 2. FIGS. 4A and 4B show an example of the arrangement of square pyramid-type needle portions 1 and frustum portions 2. FIGS. 5A and 5B show an example of the arrangement of a combination type in which the cone-type needle portions 1 are combined with the square pyramid-type frustum portions 2.

The cone type used herein means that the needle 4 has a conical shape, the body part 5 has a cylindrical shape, and the frustum portion 2 has a circular truncated conical shape. On the other hand, the square pyramid type means that the needle 4 has a square pyramidal shape, the body part 5 has a square columnar shape, and the frustum portion 2 has a truncated square pyramidal shape. The combination type used herein basically means that the cone-type needle portions 1, in which the needle 4 has a conical shape and the body part 5 has a cylindrical shape, are combined with the frustum portions 2 having a truncated square pyramidal shape.

FIG. 2A is a perspective view of an example of the arrangement of the cone-type needle portions 1 and frustum portions 2, and FIG. 2B is a plan view of the example of the arrangement of the cone-type needle portions 1 and frustum portions 2. Adjacent frustum portions 2 are formed so that the perimeters (circumferences) of larger bases of the frustum portions 2 are in contact with each other, as shown in FIGS. 2A and 2B.

Referring to FIGS. 3A and 3B, the cone-type frustum portions 2 are conceivably arranged in either of the following ways: Each frustum portion 2 is in contact with four other frustum portions 2 as shown in FIG. 3A or each frustum portion 2 is in contact with six other frustum portions 2 as shown in FIG. 3B. The arrangement shown in FIG. 3B, in which each frustum portion 2 is in contact with six other frustum portions 2, is preferable because the needle portions 1 and the frustum portions 2 can be arranged more densely over the sheet portion 3. The reason for this is that arranging the needle portions 1 and the frustum portions 2 densely allows the area of the sheet portion 3 to be reduced but a necessary amount of drug to still be supplied.

The next description will be made with reference to FIGS. 4A and 4B. FIG. 4A is a perspective view of an example of the arrangement of square pyramid-type needle portions 1 and frustum portions 2, and FIG. 4B is a plan view of the example of the arrangement of the square pyramid-type needle portions 1 and frustum portions 2. Each frustum portion 2 is in contact with eight other frustum portions 2, as shown in FIGS. 4A and 4B. The arrangement allows the needle portions 1 and the frustum portions 2 to be arranged more densely, whereby the area of the sheet portion 3 can be reduced but a necessary amount of drug can still be supplied.

The above examples have been described with reference to the cone type and the square pyramid type. The arrangement is, however, not limited thereto. Each needle 4 can arbitrarily have the conical shape or the pyramidal shape. Each body part 5 can arbitrarily have the cylindrical shape or the rectangular columnar shape. Each frustum portion 2 can arbitrarily have the truncated conical shape or pyramidal shape. That is, the needle 4, the body part 5, and the frustum portion 2 can have respective shapes arbitrarily chosen from the shapes described above and can be combined.

Further, the pyramidal shape, the rectangular columnar shape, and the truncated pyramidal shape (pyramidal frustum shape) described above can be based not only on a square shape but also on an arbitrary polygonal shape. When the pyramidal shape, the rectangular columnar shape, and the truncated pyramidal shape are based on an polygonal shape, it is also preferable to employ a closest packing arrangement (which contains the largest number of frustum portions per unit area), that is, an arrangement that allows each frustum portion 2 having a truncated pyramidal shape to be in contact with the largest number of frustum portions.

The next description will be made with reference to FIGS. 5A and 5B. FIG. 5A is a perspective view of an example of the arrangement of combination-type needle portions 1 and frustum portions 2, and FIG. 5B is a plan view of the example of the arrangement of the combination-type needle portions 1 and frustum portions 2. Each frustum portion 2 is in contact with eight other frustum portions 2, as shown in FIGS. 5A and 5B. The arrangement allows the needle portions 1 and the frustum portions 2 to be arranged densely, whereby the area of the sheet portion 3 can be reduced but a necessary amount of drug to still be supplied.

Each body part 5 having a cylindrical shape is connected to the upper surface of the corresponding frustum portion 2 having a truncated square pyramidal shape and a square bottom surface. The upper surface of the frustum portion 2 has substantially the same size as the bottom surface of the cylindrical body part 5. Each conical needle 4 is connected to the corresponding cylindrical body part 5.

Each combination-type frustum portion 2 can have an arbitrary polygonal pyramidal shape instead of a square pyramidal shape. Since a closest packing arrangement (which contains the largest number of frustum portions per unit area) is preferable, a square pyramid, a hexagonal pyramid, or a triangular pyramid is preferable. In particular, a truncated square pyramid is most preferable because a mold can be readily produced. Since a closest packing arrangement prevents flat portions from being formed over the surface of the mold, the drug will not be left in portions other than areas where the needle portions are formed.

In each combination-type needle portion 1, the needle 4 has a conical shape and the body part 5 has a cylindrical shape. The needle portion 1 having the shape described above is more readily inserted into a skin than a pyramidal-type needle portion. Each needle portion 1 may have a forward converging shape (tapered shape) or a pencil-like shape.

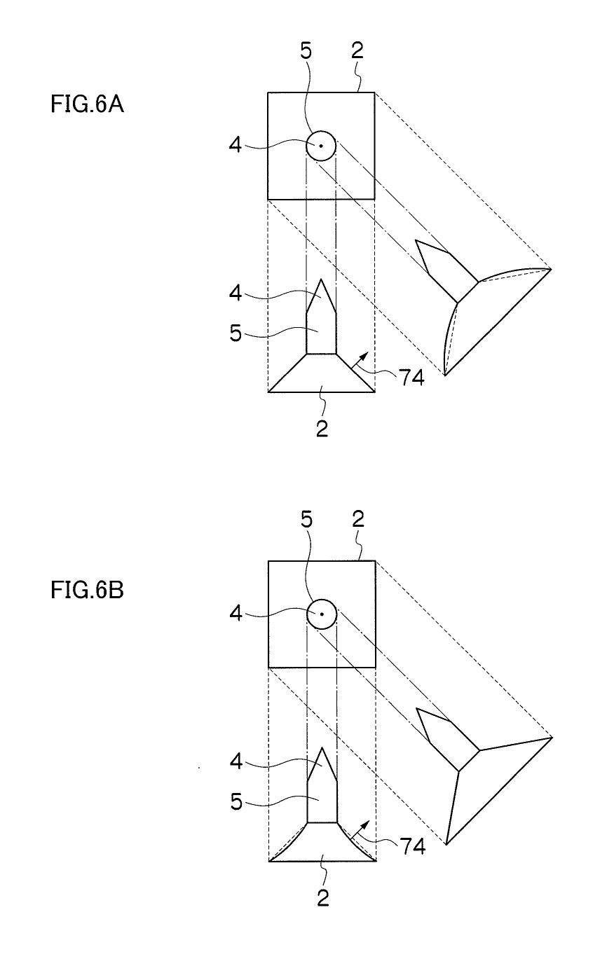

FIGS. 6A and 6B show combinations of a plan view, a front view, and a diagonal side view of two preferred combination-type forms. In the diagonal side view in FIG. 6A, the ridge of the frustum portion 2 has a slight convex shape.

In the front view in FIG. 6B, the ridge of the frustum portion 2 has a slight concave shape or slightly rounded shape.

In either of the two preferred combination-type forms, when the frustum portion 2 is placed in a horizontal plane, a normal vector 74 to the external surface of the frustum portion 2, except the region on which the needle portion 1 is formed, is not parallel to the vertical direction. This means that the frustum portion 2 has no horizontal surface when the needle portion 1 and the frustum portion 2 are viewed from above. Since no horizontal surface is formed, the drug is not left on the frustum portion 2 but is injected into the needle portion 1.

### <How Needle Array Transdermal Absorption Sheet Works>

How the needle array transdermal absorption sheet according to the present invention works will next be described with reference to FIG. 1. The needle array transdermal absorption sheet according to the present invention has a structure in which each needle portion 1 having a pencil-like shape is supported by the corresponding frustum portion 2 having a frustum shape (a truncated conical shape and a truncated pyramidal shape are referred to as frustum shapes), as shown in FIG. 1.

Since each frustum portion 2 spreads out wide (backward diverging shape) from the portion connected to the needle portion 1 toward the portion connected to the sheet portion 3, the stress produced when the needle portion 1 is inserted into the skin 100 can be distributed over a broad area in the sheet portion 3. That is, the frustum portion 2, which is in a frustum shape, can stably support the needle portion 1 without sideways movement when the needle portion 1 is inserted into the skin 100.

Each needle portion 1 has a pencil-like shape. That is, each needle portion 1 includes the needle 4 having a conical or pyramidal shape and the body part 5 having a cylindrical or rectangular columnar shape. As a result, each needle portion 1 has an advantage of being readily inserted into the skin 100 but resisting coming off the skin 100. That is, the needle 4 having a conical or pyramidal shape allows the needle portion 1 to be readily inserted into the skin 100, and the body part 5 having a cylindrical or rectangular columnar shape prevents the needle portion 1 from readily coming off the skin 100.

The reason for this is that even if any needle portion 1 slightly comes off the skin 100 due, for example, to skin elasticity, the body part 5 having a cylindrical or rectangular columnar shape is always in contact with the skin 100 and friction between the body part 5 and the skin 100 is maintained unless the body part 5 completely comes off the skin 100.

For example, when each needle portion 1 has a conical or pyramidal shape and any needle portion 1 comes off the skin due, for example, to skin elasticity even by a small amount, a space created between the needle portion and the skin causes the needle portion to easily come off the skin because no friction is present any more. As described above, the needle portion 1 having a pencil-like shape is advantageous in that it is readily inserted into the skin but resists coming off the skin, while being problematic in that it is not held in a stable manner and the stress cannot be distributed, unlike a needle portion having a conical or pyramidal shape, and hence the needle portion 1 is prone to be bent or broken when inserted into the skin. On the other hand, in the present invention, the frustum portions 2 are provided so that the disadvantage of the pencil-like shape is solved. Since the present invention provides the structure in which each frustum portion 2 having a truncated pyramidal or conical shape supports each needle portion 1 having a pencil-like shape, the needle portion 1 can be inserted into the skin in a stable manner with the stress distributed but without being broken or bent.

On the other hand, when a needle portion 1 having no frustum portion 2 but directly connected to the sheet portion 3 is inserted into a skin, convexo concaves of the skin surface prevent the root of the needle portion 1 and the vicinity of the root from being completely inserted into the skin. As a result, the entire amount of drug in the needle portion 1 will not be absorbed in the skin, resulting in reduced action of the drug.

In contrast, since the frustum portions 2 are provided with the needle portions whose frustum shape can make convex portions of the skin 100 enter the space between adjacent frustum portions 2, as shown in FIG. 1, and thus the needle portions 1 are reliably inserted into the skin 100 to a point where the root of each of the needle portions 1 comes into contact with the skin 100. As a result, the entire amount of drug contained in the needle portions 1 can be reliably absorbed in the skin.

It is noted in the present invention that only the needle portions 1 are inserted into the skin, whereas the frustum portions 2 remain outside the skin.

Further, FIG. 1 is so drawn for ease of illustration that a space formed between adjacent frustum portions 2 and having a triangular cross-sectional shape is not filled with the skin 100 but a gap is present in the space. In practice, however, the space having a triangular cross-sectional shape will be nearly filled with the skin. In this case, the skin 100 is sandwiched between the side surfaces of adjacent frustum portions 2 that form the space having a triangular cross-sectional shape, whereby the needle portions 1 will resist coming off the skin 100.

### <Method for Manufacturing Needle Array Transdermal Absorption Sheet>

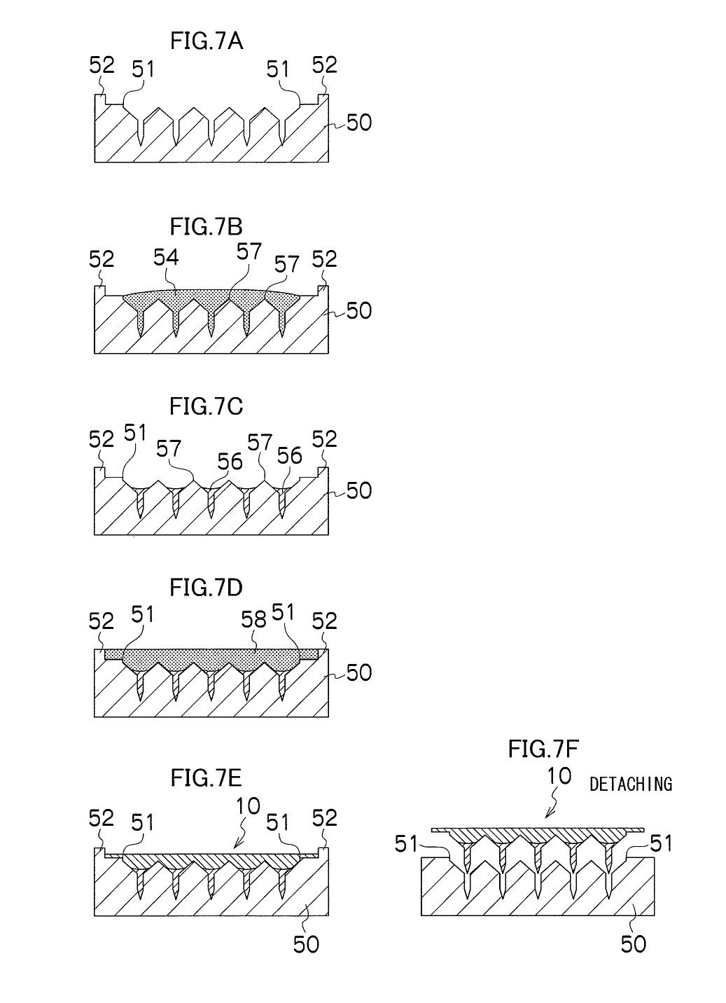

A method for manufacturing the needle array transdermal absorption sheet will next be described with reference to FIGS. 7A to 7F. FIGS. 7A to 7F show processes for manufacturing the needle array transdermal absorption sheet.

### (1) Producing Mold

A method for producing a mold for manufacturing the needle array transdermal absorption sheet will first be described. Wire-shaped metal members having a diameter of 150 μm are prepared, and the front portion in a range of 300 μm from the tip of each wire-shaped metal member is ground so that the front portion has a conical shape with the tip having a curvature radius of 5 pun.

Next, a smooth metal plate having a size of 40×40 mm is prepared, and conical recesses having a diameter of 0.5 mm and a depth of 0.3 mm are formed in the metal plate so that the conical recesses are arranged in a 10×10 matrix at intervals of 500 μm in a staggered pattern. A hole having a diameter of 160 μm is then formed at the center of each of the thus formed conical recesses, and the wire-shaped metal members with tips having been shaped as described above are so inserted through the holes that the tips protrude through the holes by 600 μm, and the metal members are fixed there. A master plate is thus produced.

The master plate and a silicone rubber (model-making RTV (Room Temperature Vulcanization) rubber manufactured by Shin-Etsu Chemical Co., Ltd.) are used to produce a reverse transferred structure, which is cut off to leave a flat portion having a size of 45×45 mm and including an array of 10×10 holes in a central portion. A mold 50 having a thickness of 5 mm is thus produced.

### (2) Preparation of Polymer Dissolved Liquid

A description will next be made of a polymer dissolved liquid used as a basic material of which the needle array transdermal absorption sheet is made and a method for preparing a liquid obtained by adding a drug to the polymer dissolved liquid. The present invention is, however, not limited to the polymer dissolved liquid and the drug described below but any biocompatible, biodegradable material can replace the polymer dissolved liquid described below and any drug according to applications can be used.

Pullulan (HAYASHIBARA SHOJI, INC.) is dissolved in water and a 15% Pullulan aqueous solution is prepared, which is then agitated at 50° C. and kept at the same temperature. The following two solutions are then prepared: a liquid obtained by adding 1% of ascorbic acid as a drug to the Pullulan solution (polymer dissolved liquid 1) and the original Pullulan solution (15% Pullulan aqueous solution dissolved at 50° C.) containing no additive (polymer dissolved liquid 2).

### (3) Manufacturing Needle Array Transdermal Absorption Sheet

A silicone sheet having a size of 40×40 mm and a thickness of 3 mm (Shin-Etsu Silico-Sheet BA grade manufactured by Shin-Etsu Finetech Co., Ltd.) is prepared, and an opening having a size of 30×30 mm is formed through a central portion of the silicone sheet. The mold produced in (1) (hereinafter simply referred to as the mold) and the silicone sheet are so positioned that the conical/cylindrical hole pattern was exposed through the opening of the silicone sheet, and the silicone sheet 52 is placed on and bonded to the mold. FIG. 7A is a cross-sectional view of the mold 50 to which the thus produced silicone sheet 52 was bonded. A step 51 is formed in the upper surface of the mold 50 along an area that forms the boundary between the frustum portions and the sheet portion. The step 51 is formed around the outermost frustum portions. The step 51 has a height of at least 0.1 mm.

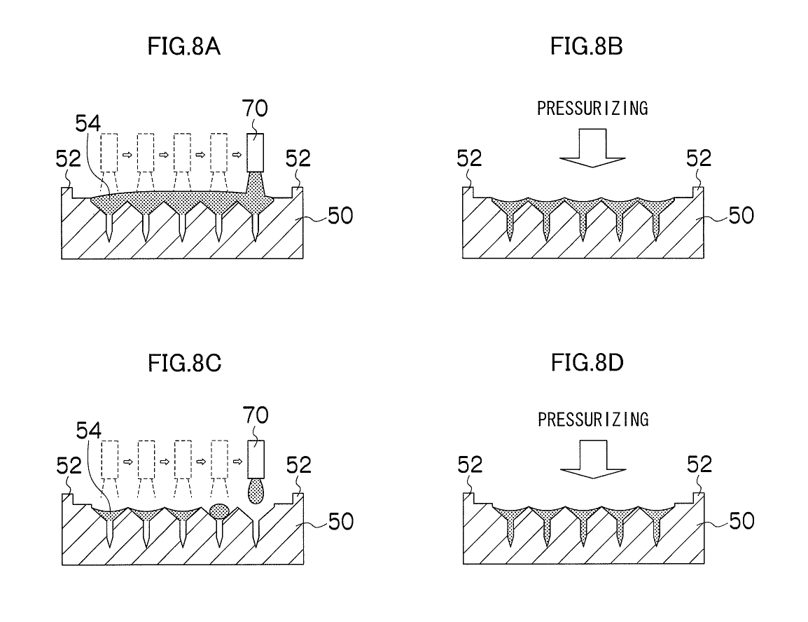

Thereafter, 1 ml of the polymer dissolved liquid 1 is dropped by using a dispenser into the mold 50 to which the silicone sheet 52 is bonded (through the opening in the silicone sheet) (FIG. 7B). In FIG. 7B, reference numeral 54 designates the dropped polymer dissolved liquid 1. Since the upper surface of the mold 50 has the step 51, the polymer dissolved liquid 1 will not spread over or wet portions other than the area where the needle portions are formed. The drug is therefore not wasted. When the dropped liquid does not tend to spread over or wet the area where the needle portions are formed, shifting the position of the dispenser 70 and dropping a minute droplet multiple times allows the liquid to be uniformly spread, as shown in FIGS. 8A and 8B.

In FIG. 8A, the dispenser 70 drops the polymer dissolved liquid 1 (54) into the area where the needle portions are formed, while the dispenser 70 is continuously moved. After the dropping process, as illustrated in FIG. 8B, the surface of the polymer dissolved liquid 1 (54) is pressurized so that the polymer dissolved liquid 1 (54) is injected into the area where the needle portions are formed.

In FIG. 8C, the dispenser 70 drops a single droplet of the polymer dissolved liquid 1 (54) into each of the holes in the area where the needle portions are formed. After the dropping process, as illustrated in FIG. 8D, the surface of the polymer dissolved liquid 1 (54) is pressurized so that the polymer dissolved liquid 1 (54) is injected into the area where the needle portions are formed.

The mold 50 into which the polymer dissolved liquid 1 has been dropped is put in a pressure container, and the internal space of the pressure container is heated to 40° C. by using a heating jacket. Pressurized air is then injected from a compressor into the pressure container, and the internal space in the pressure container is held at a pressure of 0.5 MPa for 5 minutes. Applying the pressure allows air bubbles to be removed and hence the needle portions of the mold to be filled with the polymer dissolved liquid 1 all the way to the end of each of the needle portions.

The mold is then removed from the pressure container, placed in an oven, and dried at 40° C. for 2 hours. The drying process may be performed by applying dried air having a temperature within the range of 30-60° C. to evaporate the solvent of the polymer dissolved liquid. In this case, a slight amount of solvent may be left. The drying process causes the polymer dissolved liquid 1 in a deep portion of the mold to semi-solidify (to become semisolid) (FIG. 7C). In FIG. 7C, reference numeral 56 designates the semi-solidified polymer dissolved liquid 1. It is noted that the temperature in the drying process needs to be kept at a temperature lower than the temperature at which the efficacy of the drug is compromised.

In a cross-sectional view of the needle array transdermal absorption sheet 10 according to the present invention, since the side surfaces of adjacent frustum portions 2 are in contact with each other as shown in FIG. 1, the two side surfaces form two sides of a triangle and the intersection of the two sides is a vertex of the triangle. Now, referring to FIG. 7C, the vertex of the triangle corresponds to the portion labeled with reference numeral 57 (referred to as a vertex 57) in the mold 50.

As described above, in the mold 50, since the portion between adjacent holes forms a triangle having the vertex 57, when the polymer dissolved liquid 1 is dried and the volume thereof decreases, no polymer dissolved liquid 1 will be left on the portion between the holes. That is, the entire amount of polymer dissolved liquid 1 containing an expensive drug enters the holes that will form the needle portions 1 (and part of the drug enters the portions that will form the frustum portions 2), whereby the expensive drug will not be wasted.

Each vertex 57, which cannot, of course, be an ideal point, may be a curved surface having a certain radius of curvature or a flat surface to the extent that the polymer dissolved liquid 1 is not left at the vertex. That is, the formed contact point between the side surfaces of adjacent frustum portions 2 (in a cross-sectional view) may not be an ideal point but may be a curved surface having a certain radius of curvature or a flat surface.

The amount of polymer dissolved liquid 1 to be initially injected into the mold is desirably adjusted so that when the polymer dissolved liquid 1 is dried and the volume thereof decreases, the entire amount of polymer dissolved liquid 1 is contained in the spaces corresponding to the needle portions 1. In this way, only the needle portions contain the expensive drug, whereby the entire amount of drug will be absorbed in the skin and will not be wasted.

The drug may be left on the surfaces of the areas of the mold 50 where the frustum portions are formed in some cases. The drug on that surface will be wasted because the drug will hardly penetrate the body part of a patient. It is therefore important to force the drug that adheres to the surfaces of the areas where the frustum portions are formed to flow into the areas where the needle portions are formed as much as possible.

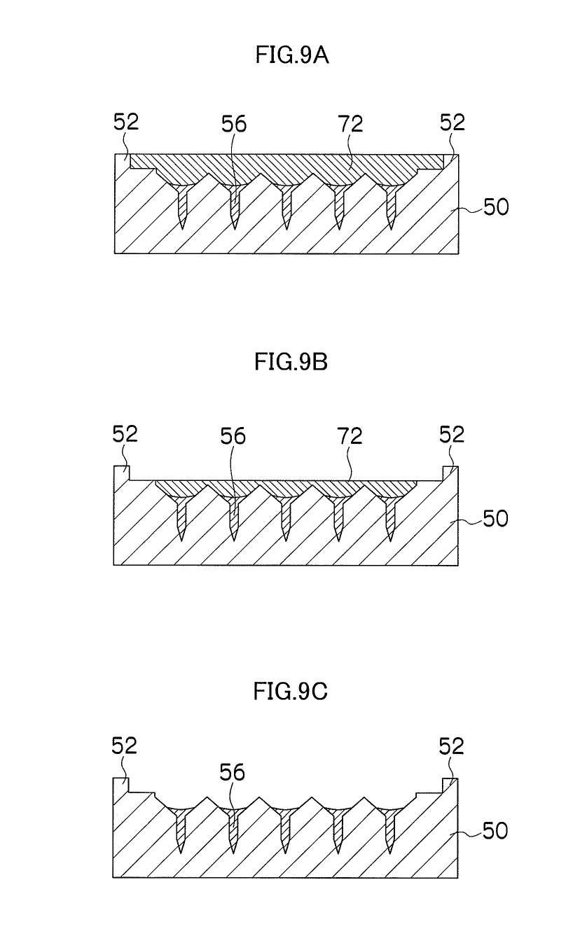

To this end, it is preferable to carry out a process shown in FIGS. 9A to 9C between the processes shown in FIGS. 7C and 7D.

The polymer dissolved liquid 1 is injected into the mold 50 and dried, and then an intermediate liquid 72 is dropped (FIG. 9A). The intermediate liquid 72 is preferably water containing no drug or a diluted drug solution. The intermediate liquid 72 is then pressurized and injected all the way to the end of each of the areas where the needle portions are formed (FIG. 9B). The intermediate liquid 72 is then dried (FIG. 9C).

In the method described above, the drug left on the surfaces of the mold 50 where the frustum portions are formed is temporarily dispersed in the intermediate liquid 72. Injecting the intermediate liquid 72 containing the drug into the areas where the needle portions are formed in the mold 50 allows a greater amount of drug to flow into the areas where the needle portions are formed. As a result, the amount of drug to be wasted will be minimized.

Alternatively, at the point of time shown in FIG. 9A, the intermediate liquid 72 is semi-dried so that the amount of water is reduced, and then the intermediate liquid 72 may be so pressurized and injected that the drug left in the areas where the frustum portions are formed flows into the areas where the needle portions are formed.

Next, 2 ml of the polymer dissolved liquid 2 is dropped into the mold (FIG. 7D) and the polymer dissolved liquid 2 is allowed to solidify at a low temperature (−5° C.). The mold is then placed in an oven and dried at 35° C. for 4 hours. After the dropping process, the mold may be pressurized in a pressure container for deaeration. In FIG. 7D, reference numeral 58 designates the dropped polymer dissolved liquid 2. The drying process may alternatively be carried out by using dried air having a temperature within the range of 30-50° C. to evaporate a sufficient amount of solvent of the polymer dissolved liquid 2. Solidifying and drying the polymer dissolved liquid 2 at a low temperature as described above prevents the drug contained in the polymer dissolved liquid 1 from diffusing into the polymer dissolved liquid 2. It is noted that the temperature in the drying process needs to be kept at a temperature lower than the temperature at which the efficacy of the drug is compromised.

In the drying process, both the polymer dissolved liquid 1 and the polymer dissolved liquid 2 solidify, and the needle array transdermal absorption sheet 10 is formed (FIG. 7E). Thereafter, the silicone sheet 52 having been placed and bonded to the mold is removed, and an adhesive tape is attached to the rear surface (the surface where no needle portions have been formed) of the needle array transdermal absorption sheet 10. The adhesive tape along with the needle array transdermal absorption sheet 10 is then detached from the mold (FIG. 7F). The needle array transdermal absorption sheet 10 is thus manufactured.

### <Evaluation of Various Dimensional Parameters of Needle Array Transdermal Absorption Sheet>

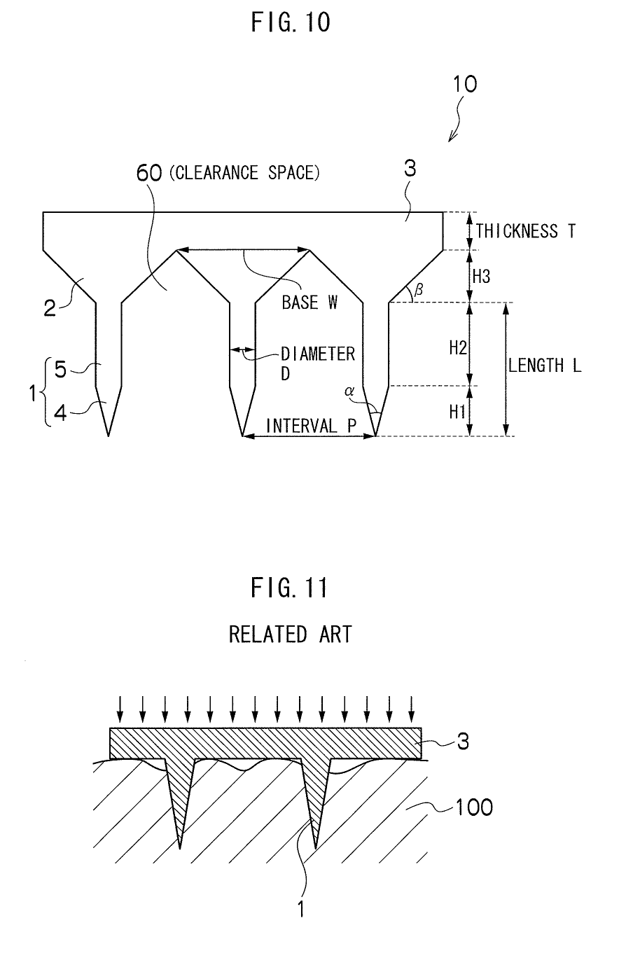

Next, dimensional parameters of a variety of portions of the needle array transdermal absorption sheet according to the present invention will be described. FIG. 10 shows reference characters that designate dimensional parameters of a variety of portions of the needle array transdermal absorption sheet.

### (1) Angle β of Frustum Portion

A description will be made of an angle β between the side surface of any frustum portion 2 and a plane parallel to the surface of the sheet portion 3. The angle β may be taken as the angle between the side surface of the frustum portion 2 and a plane perpendicular to the body part of the corresponding needle portion 1. In the following description, a person to whom the needle array transdermal absorption sheet is so attached that the needle portions 1 are inserted into the skin of the person is called a subject.

When a needle portion 1 having a very large angle β is inserted into the skin, the frustum portion 2 is also inserted into the skin. In this case, the subject feels pain. Further, when the height H3 is fixed and the angle β is too large, the area of the portion where each frustum portion 2 is connected to the sheet portion 3 becomes small. In this case, the stress produced when the needle portion 1 is inserted into the skin is not distributed in a satisfactory manner, and the needle portion 1 tends to be broken or bent or otherwise damaged. The angle β should therefore not be too large.

On the other hand, when the angle β is too small and the height H3 is fixed, the interval P becomes large. In this case, the number of needle portions 1 per unit area decreases and hence a necessary amount of drug cannot be supplied to the subject. Further, when the angle β is set at a smaller value while the interval P is not increased but is set at a fixed value, the height H3 of the frustum portion 2 becomes small. In this case, since the area of the portion where the frustum portion 2 is connected to the sheet portion 3 becomes small, the stress produced when the needle portion 1 is inserted into the skin is not distributed in a satisfactory manner, and hence the needle portion 1 is likely broken or bent or otherwise damaged.

Further, the volume of a clearance space 60, which is a triangular space in the cross-sectional view sandwiched between the two side surfaces of adjacent frustum portions 2, becomes small. In this case, when the needle portion 1 is inserted into the skin, the space that convex portions of convexo concaves of the skin enter becomes small and the convex portions of the skin push back the needle array transdermal absorption sheet 10. The needle portions 1 will therefore not be inserted into the skin to a point where the root of each of the needle portions 1 come into contact with the skin. The angle β should therefore not be too small.

In view of the circumstances described above, evaluation was performed for a variety of angles β. A needle array transdermal absorption sheet was manufactured by using the manufacturing method described above and actually attached onto a subject, and pain felt by the subject and how well the needle portions 1 were inserted into the skin were evaluated. How well the needle portions 1 were inserted into the skin was evaluated by performing direct visual inspection and measurement of the distance from the skin surface to the rear surface of the sheet portion 3. Parameters other than the angle β in the evaluation were as follows: the base W was variable; the diameter D was 0.12 mm; the interval P was variable; the angle α was 27°; H1 was 0.25 mm; H2 was 0.25 mm; H3 was 0.15 mm; and the thickness T was 0.15 mm.

Table 1 shows evaluation results.

TABLE 1

| | | How well needle portions were |
|---|---|---|
| Angle β | Pain | inserted into skin |
| 15° | No | Part of sheet is apart from skin |
| 20° | No | Part of sheet is apart from skin only |
| | | by very small amount |
| 30° | No | Sheet is not apart from skin |
| 40° | No | Sheet is not apart from skin |
| 50° | No | Sheet is not apart from skin |
| 60° | No pain but unpleasant | Sheet is not apart irom skin |
| | sensation | |
| 65° | Yes | Sheet is not apart from skin |

The results show that the angle β preferably is a value within the range of 20° to 60°, more preferably 30° to 50°.

### (2) Interval P Between Needle Portions

When the interval P, at which the needle portions 1 are arranged, is larger, the needle portions 1 are more readily inserted into the skin, whereas the interval P is smaller, the needle portions 1 more resist being inserted into the skin. When the interval P is too large, the number of needle portions 1 per unit area becomes small, and a sufficient amount of drug cannot be supplied to the subject.

The needle array transdermal absorption sheets having a variety of intervals P as a parameter were manufactured as in (1) and attached onto a subject, and how well the needle portions were inserted into the skin was evaluated. Parameters other than the interval P in the evaluation were as follows: The base W was variable; the diameter D was 0.12 mm; the angle β was 30°; the angle α was 27°; H1 was 0.25 mm; H2 was 0.25 mm; H3 was 0.02 mm; and the thickness T was 0.15 mm.

Table 2 shows evaluation results.

TABLE 2

| Interval P (mm) | Easiness to insert needle portions into skin |
|---|---|
| 0.2 | Extremely bad |
| 0.3 | Extremely bad |
| 0.5 | Satisfactory |
| 0.6 | Satisfactory |
| 1.0 | Extremely well |

As shown in Table 2, when the interval P was 0.5 mm or greater, the needle portions were inserted into the skin in a satisfactory manner. In consideration of the number of needle portions 1 per unit area, the interval P is preferably a value within the range of 0.5-0.6 mm from the viewpoint of supplying the drug.

### (3) Length L, Diameter D, Angle α, Relationship Between H1 and H2, H3, and Thickness T

Evaluation was performed for the following parameters: the length L of the needle portion 1; the diameter D of the body part 5; the angle α of the tip of the needle 4; the relationship between the length H1 of the needle 4 and the length H2 of the body part 5; the height H3 of the frustum portion 2; and the thickness T of the sheet portion 3. In the evaluation, a preferred range of each of the parameters was determined.

The needle array transdermal absorption sheets 10 having a variety of values of the parameters described above were manufactured as in (1) and attached onto a subject for evaluation. The parameters other than those to be evaluated were as follows: the base W was 0.64 mm; the diameter D was 0.12 mm; the interval P was 0.64 mm; the angle α was 27°; H1 was 0.25 mm; H2 was 0.25 mm; H3 was 0.15 mm; the thickness T was 0.15 mm; and the angle β was 30°.

Table 3 shows evaluation results.

TABLE 3

| Parameter | Preferred range | Evaluation results |
|---|---|---|
| Length L | 0.3 mm-1.5 mm | When the length L was shorter than 0.3 mm, the |
| | | needle portions were not inserted into the skin, and |
| | | the amount of drug administrated to the subject |
| | | decreased. When the length L was longer than 1.5 mm, |
| | | inserting the needle portions caused pain. |
| Needle diameter | 0.1 mm-0.3 mm | When the needle diameter D was smaller than 0.1 mm, |
| D | | it was difficult to form the needle portions 1, |
| | | and the amount of drug administrated to the subject |
| | | decreased. When the needle diameter D was |
| | | greater than 0.3 mm, the needle portions 1 resisted |
| | | being inserted into the skin and inserting the needle |
| | | portions caused pain. |
| Angle α | 20°-60° | When the angle α was smaller than 20°, it was |
| | | difficult to form the needle portions 1, and H1 was |
| | | too long. When H1 was too long, the needle |
| | | portions 1 readily came off the skin. When the |
| | | angle α was greater than 60°, the needle portions 1 |
| | | resisted being inserted into the skin. |
| Relationship | H1/H2 = ½-2 | When H1/H2 was greater than 2, the proportion of |
| between H1 and | | the body part 5 became small and the needle |
| H2 | | portions 1 readily came off the skin. When H1/H2 |
| | | was smaller than ½, the needle portion 1 had |
| | | substantially a columnar shape and resisted being |
| | | inserted into the skin. |
| H3 | 0.1 mm-0.5 mm | When H3 was smaller than 0.1 mm, the needle |
| | | portions 1 resisted being inserted into the skin to a |
| | | point where the roots thereof came into contact with |
| | | the skin. When H3 was greater than 0.5 mm, the |
| | | interval became large, resulting in decrease in the |
| | | amount of drug administered to the subject. |
| Thickness T | 0.1 mm-0.4 mm | When the thickness T was smaller than 0.1 mm, the |
| | | sheet was too thin and was not handled |
| | | satisfactorily. When the thickness T was greater |
| | | than 0.4 mm, the sheet was not flexible enough or |
| | | fit well with the skin surface. |

As shown in Table 3, the parameters of the length L, the diameter D, the angle α, the relationship between H1 and H2, H3, and the thickness T, have respective appropriate values. These values were not determined simply in order to find optimum conditions. Instead, the present inventors have intensively conducted studies, found unknown effects associated with the parameters, and determined ranges within which the effects take place. The effects described in Table 3 take place by manufacturing the needle array transdermal absorption sheet in such a way that the parameters fall within the preferred ranges thereof described in Table 3.

What is claimed is:

1. A method for manufacturing a needle array transdermal absorption sheet, comprising:
injecting a first polymer dissolved liquid containing a drug into a space of a mold, the space having a shape identical to a shape of the needle array transdermal absorption sheet, the mold including a plurality frustum portions, wherein each of the plurality of frustum portions includes has a large perimeter base and a small perimeter base, wherein each small perimeter base is connected to a needle portion to form a plurality of needles, and wherein the large perimeter bases of the plurality of frustum portions are disposed adjacently on a plate-shaped sheet;
filling the space in the mold with the first polymer dissolved liquid to an end of the space by pressurizing the mold into which the first polymer dissolved liquid has been injected to remove air bubbles from a portion where the first polymer dissolved liquid has been injected;
drying and shrinking the first polymer dissolved liquid by heating the first polymer dissolved liquid so that the first polymer dissolved liquid in the space in the mold is positioned closer to portions of the mold corresponding to the needle portions than portions of the mold corresponding to peripheries of the large perimeter base of the frustum portions;
injecting a second polymer dissolved liquid into the space in the mold;
solidifying the first polymer dissolved liquid and the second polymer dissolved liquid by heating the first polymer dissolved liquid and the second polymer dissolved liquid; and
separating and removing the solidified first and second polymer dissolved liquids from the mold.

2. A method for manufacturing a needle array transdermal absorption sheet according to claim 1, wherein the each of the needle portions has a pencil-like shape.

3. A method for manufacturing a needle array transdermal absorption sheet according to claim 1,
wherein an angle β between a side surface of each of the frustum portions and a plane parallel to a planar surface of the plate-shaped sheet falls within a range of 20° to 60°.

4. A method for manufacturing a needle array transdermal absorption sheet according to claim 1,
wherein a height of each of the frustum portions falls within a range of 0.1 mm to 0.5 mm.

5. A method for manufacturing a needle array transdermal absorption sheet according to claim 1,
wherein each of the needle portions has a tapered shape comprising a circular conical portion at one end and a cylindrical columnar shape at another end which is adjacent to the frustum, and
each of the frustum portions has a truncated pyramidal shape.

6. A method for manufacturing a needle array transdermal absorption sheet according to claim 2,
wherein each of the needle portions having a pencil-like shape includes a circular conical portion at one end and a cylindrical columnar shape at another end which is adjacent to the frustum, and
each of the frustum portions has a truncated pyramidal shape.

7. A method for manufacturing a needle array transdermal absorption sheet according to claim 1,
wherein when the large perimeter bases of the frustum portions are placed in a horizontal plane, a normal vector to each of the frustum portion is not parallel to a vertical direction.
